(19) United States
(12) Patent Application Publication
(10) Pub. No.: US 2026/0130886 A1
(43) Pub. Date: May 14, 2026

(54) EXTRUDED SOFT CHEW CONTAINING A NON-ANIMAL BASED PALATANT AND ACTIVE PHARMACEUTICAL INGREDIENTS

(71) Applicant: VIRBAC, Carros (FR)

(72) Inventors: David CORVAISIER, BIOT (FR); Thomas PEZE, Carros (FR)

(73) Assignee: VIRBAC, Carros (FR)

(21) Appl. No.: 18/947,429

(22) Filed: Nov. 14, 2024

(51) Int. Cl. A61K 31/365; A61K 9/00; A61K 31/4985; A61K 47/10; A61K 47/26; A61K 47/36; A61K 47/38; A61K 47/44; A61K 47/46

(52) U.S. Cl. CPC A61K 31/365; A61K 9/0056; A61K 31/4985; A61K 47/10; A61K 47/26; A61K 47/36; A61K 47/38; A61K 47/44; A61K 47/46

(57) ABSTRACT

A veterinary extruded soft chew has as active pharmaceutical ingredients (APIs), milbemycin oxime or one of its salts, and praziquantel or one of its salts. The APIs are uniformly dispersed in a matrix having one or more fillers, a polyethylene glycol (PEG), a non-animal based palatant, a disintegrant, a humectant, an oil, water, and preservatives. Neither the milbemycin oxime or one of its salts, nor the praziquantel or one of its salts is coated, granulated or physically separated from the other APIs within the matrix. More importantly, the soft chew does not comprise any palatant of animal origin.

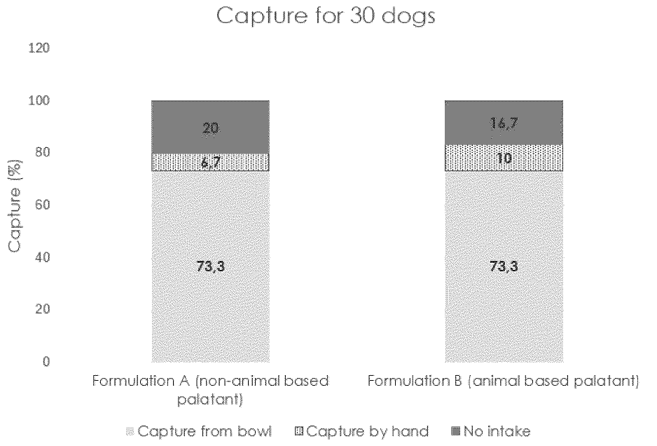

### FIELD OF THE INVENTION

The present invention relates to a soft chew comprising at least a non-animal based palatant and two active pharmaceutical ingredients (APIs): milbemycin oxime (MBO) or one of its salts, and praziquantel or one of its salts, for the administration to pets, a process for manufacturing said soft chew, a soft chew directly obtaining by a cold extrusion process and the use of such soft chew.

### BACKGROUND TO THE INVENTION

Chewable pharmaceutical dosage units, such as soft chews, are known and have been commercialized for pets, mainly dogs and/or cats that are carnivorous. Formulating a drug into a chewable dosage form can enhance acceptance among animal patients that typically resist swallowing hard tablets or capsules, potentially even leading them to willingly consume the medication on their own.

The stability of active pharmaceutical ingredients (APIs) in soft chews for pets is a critical consideration for ensuring the effectiveness and safety of the product over its shelf life. The key factors to consider are notably:
Formulation: The compatibility of APIs with the other ingredients in the soft chew formulation is essential. Ingredients should not react with each other in a way that degrades the API.
Water Content: Soft chews typically have higher water content than tablets or capsules. Excessive water can lead to hydrolysis or degradation of certain APIs. Controlling the water activity in the product is crucial for stability.
Temperature Sensitivity: APIs can be sensitive to temperature fluctuations. Storing soft chews in a cool, dry place can help maintain their stability. It's important to understand the specific temperature range in which the API remains stable.
pH Levels: The pH of the soft chew can affect the stability of the API. Some APIs are stable only within a specific pH range. Formulators must adjust the pH of the chew to optimize the stability of the API.
Packaging: Packaging plays a significant role in protecting the API from environmental factors like light, oxygen, and moisture. Using appropriate packaging materials that provide a barrier against these elements can enhance the stability of the API.
Preservatives: Adding preservatives can help prevent microbial growth, which can affect the stability of the API. However, preservatives must be compatible with both the API and the pet's health. Some antioxidants may also act as preservatives.
Shelf Life Testing: Conducting rigorous stability testing under various conditions (temperature, humidity, light) helps determine the shelf life of the product. This testing typically follows guidelines provided by regulatory authorities such as the FDA or EMA.
Manufacturing Process: The process used to manufacture the soft chews can impact API stability. Processes that involve high temperatures or shear forces can potentially degrade sensitive APIs.
Analytical Methods: Developing reliable analytical methods to monitor the stability of the API throughout the shelf life of the product is essential. These methods should detect any degradation products that may form.
Regulatory Compliance: Ensuring that the formulation and stability of the product comply with regulatory requirements is crucial for market approval and consumer safety.

By addressing these factors, manufacturers can optimize the stability of APIs in soft chews, ensuring that the product remains effective and safe for its intended use over its entire shelf life.

The acceptance of a pharmaceutical by a pet is determined mainly by its odour and taste. The palatability of a chewable medicinal product is also largely determined by its texture and the mouthfeel generated. In the case of dogs, soft structures are preferred considerably to hard and brittle structures such as tablets.

Palatants are additives used in pet foods, including soft chews, to enhance their flavor and aroma, making them more appealing to pets. The palatants should be compatible with other ingredients, including active pharmaceutical ingredients (APIs), to avoid any adverse reactions or degradation. CN114796140 discloses soft chews comprising milbemycin and praziquantel, in which 25 to 35% of a meat powder is present. Meat powder is an animal-based palatant, which can pose potential allergenic risks to pets, particularly those with food sensitivities. These risks include allergic reactions, which can manifest in various symptoms such as skin irritation, gastrointestinal distress, or respiratory issues. Additionally, animal-based palatants may be susceptible to microbial contamination, which can lead to product deterioration and potential health risks to pets. Consequently, while these animal-based palatants can significantly improve the palatability and compliance of veterinary medications, it's essential to consider the potential for adverse reactions, notably hypersensitivity and allergic reactions in susceptible animals. To mitigate risks associated with food allergies, it is of utmost importance to develop hyposensitive soft chew formulations. Meat powder presents several other significant challenges. This component may include various meat by-products, making it difficult to ensure precise and uniform traceability. Animal-based palatants like meat powder are also susceptible to microbial contamination, which can lead to product deterioration and potential health risks for animals. Therefore, although these palatants can significantly improve the palatability and adherence to veterinary medications, it is crucial to consider the potential for adverse reactions, most notably hypersensitivity and allergic reactions in susceptible animals. WO2019/021191 discloses starch-free soft-chews comprising milbemycin and praziquantel. After two months at 25° C. and 60 RH, the amount of milbemycin oxime in the soft-chews has already dropped below 95% of the original content.

WO2021/013825 explains that conventional soft chew formulation, namely the one of the Bravecto soft chew, cannot be used for combination products comprising fluralaner and a macrocyclic lactone due to the degradation of the latter. WO2021/013825 therefore teaches to physically separate the macrocyclic lactone from the other active to improve its stability.

WO202251478 discloses tablets obtained by compaction of coated granules comprising praziquantel and moxidectin and granules containing sarolaner and in some cases pyrantel.

Hence, in prior art, it is suggested to physically separate macrocyclic lactones from the other active ingredients, in the presence or not of a stabilizing agent in order to improve its stability.

The technical problem underlying the present invention is therefore to provide a chewable veterinary dosage form that permits the stability of APIs, more particularly milbemycin oxime and praziquantel and at the same time that is palatable for pets and hypoallergenic.

The technical problem is solved by the embodiments provided herein below and as characterized in the appended claims.

In particular, the technical problem is solved by the use of a cold extrusion process for the manufacture of the chewable pharmaceutical dosage units. Cold extrusion technology is an innovative process in the production of pet treats and supplements, particularly for soft chews. Unlike conventional extrusion, this technique operates at a controlled and lower temperature, providing significant advantages for preserving the nutritional and functional qualities of sensitive ingredients.

This method is especially suited for soft chews according to the invention, which are designed to be easily consumed by pets while maintaining the integrity of their ingredients.

The core principle of cold extrusion lies in avoiding high temperatures. In contrast to traditional thermal extrusion, which typically operates at elevated temperatures ranging from about 90° C. to 150° C. to facilitate the mixing, cooking, and shaping of products, the cold extrusion process maintains a temperature threshold that does not exceed 60° C. For applications requiring the preservation of sensitive active ingredients—such as vitamins, botanical extracts, or other thermolabile components—the temperature may be specifically controlled within a lower range, typically from approximately 20° C. to 60° C. This range allows for optimal texture formation and product integrity without degrading or denaturing heat-sensitive compounds.

Within this low-temperature extrusion method, specific sub-ranges can be applied depending on ingredient stability requirements. For example, temperatures between 40° C. and 60° C. are suitable for forming cohesive soft chew textures while retaining ingredient functionality, particularly for moderately heat-sensitive components. In cases where highly thermosensitive ingredients are present, the extrusion temperature can be further reduced, from about 20° C. to 40° C., preferably from about 25° C. to 35° C., more preferably up to 30° C., to ensure that these ingredients retain their active properties. This process relies on high mechanical pressure to shape the final product without denaturing or degrading its ingredients.

For soft chews, cold extrusion offers several key benefits.

First, it preserves active ingredients, such as vitamins, minerals, and certain botanical extracts, which can be heat-sensitive. By processing at lower temperatures, cold extrusion minimizes the risk of these active components losing efficacy, resulting in a stable, high-quality product. This preservation is essential for products that aim to deliver specific health benefits to pets.

Additionally, cold extrusion enhances palatability and texture.

Low temperatures help maintain the natural flavors and scents of the ingredients, making the soft chews more appealing to pets, which is crucial for ensuring acceptance. The cold extrusion process also achieves an ideal chewy texture without requiring excessive gelling agents or additives, making the product easy to consume and enjoyable for pets.

### SUMMARY OF THE INVENTION

In a first aspect, the invention relates to a veterinary extruded soft chew comprising:
as active pharmaceutical ingredients (APIs)
a) milbemycin oxime or one of its salts, and
b) praziquantel or one of its salts,
   said APIs being uniformly dispersed in a matrix comprising:
one or more fillers, selected from starch, sugar, microcrystalline cellulose or mixtures thereof,
a polyethylene glycol (PEG) having a molecular weight of 3000 to 8000 g/mol,
a non-animal based palatant,
a disintegrant selected from croscarmellose sodium, sodium carboxymethyl cellulose, sodium starch glycolate or mixtures thereof,
a humectant selected from glycerol, sorbitol, propylene glycol, liquid polyethylene glycol, liquid paraffin or mixtures thereof,
an oil selected from canola oil, corn oil, soybean oil, safflower oil, cottonseed oil, soybean oil, olive oil or mixtures thereof,
water, and
preservatives,
   wherein neither the milbemycin oxime or its salts, nor the praziquantel or its salts is coated, granulated or physically separated from the other APIs within the matrix and, wherein the soft chew does not comprise any palatant of animal origin.

In a second aspect, the invention relates to a soft chew, directly obtained by a cold extrusion process, said soft chew comprising as active pharmaceutical ingredients (APIs):
a) milbemycin oxime or one of its salts, and
b) praziquantel or one of its salts, said APIs being uniformly dispersed in a matrix comprising:
one or more fillers, selected from starch, sugar, microcrystalline cellulose or mixtures thereof,
a polyethylene glycol (PEG) having a molecular weight of 3000 to 8000 g/mol,
a non-animal based palatant,
a disintegrant selected from croscarmellose sodium, sodium carboxymethyl cellulose, sodium starch glycolate or mixtures thereof,
a humectant selected from glycerol, sorbitol, propylene glycol, liquid polyethylene glycol, liquid paraffin or mixtures thereof,
an oil selected from canola oil, corn oil, soybean oil, safflower oil, cottonseed oil, soybean oil, olive oil or mixtures thereof,
water, and
preservatives,
   said cold extrusion process comprising the steps of
i. : mixing directly and intimately the ingredients, in a blinder and/or an extruder wherein neither the milbemycin oxime or its salts, nor the praziquantel or its salts is coated, granulated or physically separated from the other APIs within the blend, at a temperature not exceeding 60° C.,
ii. : extruding the final blend by subjecting the mixture to high mechanical pressure to form a cohesive matrix without thermal cooking;
iii. extruding and forming the mixture into a soft chew form, said soft chew exhibiting a tender texture and retaining the active properties of the APIs, characterized by a water content of up to 10% and an active ingredient activity of at least 95% relative to initial values prior to extrusion.

In another aspect, the invention relates to a cold extrusion process for the manufacture of said extruded soft chew, comprising the following steps:
i. mixing, a blinder and/or an extruder, directly and intimately at least the following ingredients:
   a) milbemycin oxime or one of its salts, and b) praziquantel or one of its salts,
   one or more fillers, selected from starch, sugar, microcrystalline cellulose or mixtures thereof,
   a polyethylene glycol having a molecular weight of 3000 to 8000 g/mol,
   a non-animal based palatant,
   a disintegrant selected from croscarmellose sodium, sodium carboxymethyl cellulose, sodium starch glycolate or mixtures thereof,
   a humectant selected from glycerol, sorbitol, propylene glycol, liquid polyethylene glycol, liquid paraffin or mixtures thereof,
   an oil selected from canola oil, corn oil, soybean oil, safflower oil, cottonseed oil, soybean oil, olive oil or mixtures thereof,
   water, and
   preservatives,
      wherein neither the milbemycin oxime or its salts, nor the praziquantel or its salts is coated, granulated or physically separated from the other APIs within the blend
ii. extruding the final blend, and
iii. forming a soft chew.

In another aspect, the invention relates to said soft chew for use as a medicament for non-human animals, preferably dogs and/or cats.

More particularly, the soft chew according to the present invention is used in the treatment and/or prevention of parasitic infestations in pets.

### Benefits Provided

There is no teaching in prior art of a soft-chew comprising milbemycin oxime and praziquantel in the same matrix, wherein the macrocyclic lactone is stable.

This result was unexpected in view of preliminary compatibility studies with binary mixtures of milbemycin oxime and different palatants. Indeed, this study showed that after 60 days at 50° C. in a dry oven, the stability of milbemycin oxime was not significantly affected by the nature of the palatant.

It was however found that, in the soft-chew matrix of the invention, the presence of a palatant of animal origin, such as poultry liver powder, induces the degradation of milbemycin oxime to unacceptable levels (e.g. about 88% of the initial amount after 3 months at 40° C./75% RH and about 78% after 6 months at 40° C./75% RH).

In addition, the use of poultry liver powder strongly influenced the characteristics of the soft chew. it was indeed found that after 3 months of storage at 25° C./60% RH or 40° C./75% RH, the soft chew became too hard (its hardness almost doubling) and had an unacceptable disintegration time (almost doubled as well).

In contrast, a soft chew containing the same excipients in the same proportions had an acceptable disintegration time during the whole storage time. The soft chew hardened slightly but its hardness remained acceptable after 3 months of storage at 25° C./60% RH and 40° C./75% RH.

The activity of an active pharmaceutical ingredient (API) is defined as its ability to exert the desired biological or pharmacological effect in the final product. This activity primarily depends on the chemical and functional integrity of the active pharmaceutical ingredients after processing, which is particularly important in processes like cold extrusion, where the goal is to preserve efficacy despite the processing.

Activity can be measured by the stability of the ingredient after the manufacturing process, by evaluating the percentage of degradation compared to the initial amount of introduced ingredient. An activity of 95% compared to the initial state means that 95% of the active ingredient has remained stable and biologically functional.

### DESCRIPTION OF THE FIGURE

The FIGURE is a histogram that illustrates a palatability test, i.e., the comparison of the capture rate between soft chews containing non-animal based palatants and those containing animal-based palatants, tested on 30 dogs.

### DETAILED DESCRIPTION OF THE INVENTION

Unless otherwise stated, all percentages are expressed as weight percent.

As used herein, when a range of values is expressed as “comprised between x and y”, this range includes the end-points x and y.

In a first aspect, the present invention relates to a veterinary extruded soft chew. More particularly, the present invention relates to a palatable, chewable veterinary dosage form presented as an extruded soft chew.

In the sense of the present invention, a soft chew is a formed body exhibiting a meat-like or stock cube soft texture that can be chewed readily by animals such as dogs and/or cats while providing a pleasant mouthfeel to the animals. The term soft texture as used herein refers to a texture, which holds its shape until certain levels of pressure are applied.

The size and shape of the soft chew may be adapted to the type of animal (dog or cat) and to the size of the animal. The weight of the soft chew typically ranges from 0.1 g to 20 g, preferably from 0.5 g to 10 g and most preferably from 1 g to 7 g. In particular embodiments, the soft chews weight 0.5, 0.6, 1, 2, 3, 3.3, 4, 5, 6, 6.6 or 7 g.

There exist several parameters that can be used to define the texture of a soft chew, such as firmness, softness, hardness, toughness, springiness, break force, ripeness, skin strength, work of penetration, work of extrusion. The texture of the soft chew can be determined using methods and apparatuses well known in the art, in particular texture analyzers.

The term “extruded soft chew” refers to a soft chew that is obtained by extrusion, i.e. mixing and forming the soft chew are performed with an extruder. All the ingredients of the soft chew may be charged in an inlet port, mixed in the extruder during conveyance to the discharge port or the solid ingredients charged in the inlet port and the liquids added through additional inlet ports located on the barrel of the extruder. The soft chew is shaped (formed) by the die of the extruder and the extrudate cut into soft chews of appropriate size and weight.

There are two main types of blending processes used in the production of soft chews: wet blending and dry blending.

In a wet blending process, all solid and liquid ingredients, including active pharmaceutical ingredients (APIs), excipients, and flavorings, are combined and mixed together to form a homogenous blend. This mixture is then directly fed into an extruder. The extruder heats and melts the mixture, forcing it through a die to form the desired shape of the soft chew.

In the dry blending process, solid ingredients such as powders and granules are initially mixed together to form a dry blend.

Liquid ingredients, including water, flavorings, and other additives, are then added to the dry blend. This moistened mixture is subsequently fed into an extruder and processed to form the desired shape of the soft chew.

For the purpose of the invention, the term “extruder” refers to mono-, twin- and multi-screw extruders, planetary roller extruders, vacuum filler extruders and cascade extruders, extruders equipped with a pusher, without being limited to these extruder variants. Preferably, the extruder is an extruder equipped with a pusher.

According to the invention, the soft chew comprises as active pharmaceutical ingredients (APIs):
a) milbemycin oxime or one of its salts, and
b) praziquantel or one of its salts.

The term “Active Pharmaceutical Ingredient” (API) refers to a substance or combination of substances within a pharmaceutical drug that is biologically active. APIs are the primary components responsible for the therapeutic effects of the drug. In other words, they are the ingredients that produce the intended effects on the body to treat or prevent diseases or conditions.

Milbemycin oxime is a veterinary medication from the group of milbemycins, used as a broad spectrum antiparasitic. It is active against worms (anthelmintic) and mites (miticide).

Milbemycin oxime works by disrupting neurotransmission in parasites, which leads to their paralysis and death. It binds to glutamate-gated chloride ion channels in the nerve and muscle cells of invertebrates, causing an influx of chloride ions that hyperpolarizes the cell membrane. This hyperpolarization leads to paralysis and death of the parasite.

Milbemycin oxime is particularly used for heartworm prevention, intestinal worms and mites.

Praziquantel is a medication used to treat a number of types of parasitic worm infections in mammals, birds, amphibians, reptiles, and fish.

Praziquantel increases the permeability of the membranes of parasitic worms to calcium ions. This leads to contraction and paralysis of the worms' musculature, dislodging them from their site of attachment in the host and allowing them to be removed by the host's immune system or expelled from the body.

Praziquantel is particularly used for schistosomiasis, tapeworm Infections and other parasitic infections.

Advantageously, the soft chew according to the invention comprises 1.1-11 wt. % of APIs relative to the total weight of the soft chew, more preferably 3-5 wt. % of APIs relative to the total weight of the soft chew.

The said APIs are uniformly dispersed in a matrix comprising:
one or more fillers, selected from starch, sugar, microcrystalline cellulose or mixtures thereof,
a polyethylene glycol (PEG) having a molecular weight of 3000 to 8000 g/mol,
a non-animal based palatant,
a disintegrant selected from croscarmellose sodium, sodium carboxymethyl cellulose, sodium starch glycolate or mixtures thereof,
a humectant selected from glycerol, sorbitol, propylene glycol, liquid polyethylene glycol, liquid paraffin or mixtures thereof,
an oil selected from canola oil, corn oil, soybean oil, safflower oil, cottonseed oil, soybean oil, olive oil or mixtures thereof,
water, and
preservatives,
   wherein neither the milbemycin oxime or its salts, nor the praziquantel or its salts is coated, granulated or physically separated from the other APIs within the matrix and, wherein the soft chew does not comprise any palatant of animal origin.

The Applicant has indeed found out that even if granulation of API is a good way to protect Milbemycin the process is however more complex and implicates more steps, i.e. the technique of granulated API complicates the fabrication process. By integrating the API directly into the soft chew matrix without granulation or other physical separation from the other APIs within the matrix, the Applicant has eliminated the need for these additional processing steps. As the APIs are not physically separated by granulation or other means, there is no longer any need for specific preparations associated with granulation techniques. This approach not only simplifies the manufacturing process but also potentially reduces production costs and improves scalability. It also minimizes the risk of inconsistent dosing that can occur in granulation when active ingredients are not evenly distributed throughout the product. This streamlined process enhances the product's market competitiveness by improving its manufacturability and ensuring consistent quality. The term “fillers” refers to excipients or diluents, which are inert substances added to a drug formulation to provide volume, stability of the formulation, enhance its flow properties and aid in the drug manufacturing process. Fillers are essential components in the production of various dosage forms such as soft chews. They are selected based on their physicochemical properties, such as particle size, flowability, and compressibility, to ensure optimal performance.

In one embodiment, said one or more fillers is a mixture of starch, sugar and microcrystalline cellulose.

Starch is used as a diluent in the formula and is commonly used in soft chews. It allows the soft chew formula to reach the required texture.

Microcrystalline cellulose is also used as a diluent in the formula. The particular grade of the Microcrystalline Cellulose has been chosen to have a better flowability of the dry blend.

It allows to have a correct texture of the chew and a good extrusion, its proportion is linked with the other dry powder.

In a preferred embodiment, the fillers comprise 19-23 wt. % of starch of total weight of the soft chew, 3-6 wt. % of sugar of total weight of the soft chew and 3-6 wt. % microcrystalline cellulose of total weight of the soft chew.

Advantageously, the soft chew according to the invention comprises 23-60 wt. % of fillers relative to the total weight of the soft chew, more preferably 25-35 wt. % of fillers relative to the total weight of the soft chew.

A polyethylene glycol (PEG) having a molecular weight of 3000 to 8000 g/mol is defined as being a high molecular weight PEG. High molecular weight polyethylene glycols (PEGs), such as PEG 3350 and PEG 8000, are frequently used in the formulation of soft chews in veterinary medicine and nutraceuticals for several beneficial reasons. High molecular weight PEGs contribute to the desirable texture and consistency of soft chews. They act as binders and plasticizers, helping to create a chewable, flexible product that is easy for animals to consume. These PEGs help stabilize active ingredients within the soft chew matrix, protecting them from degradation due to environmental factors such as moisture and heat. This ensures the efficacy of the active ingredients throughout the product's shelf life. PEGs can enhance the palatability of soft chews by contributing to a smooth and consistent mouthfeel. This is crucial for ensuring that animals readily accept and consume the medication or supplement without resistance. In some formulations, high molecular weight PEGs can be used to control the release of active ingredients. This can be particularly useful in creating products that provide a sustained release of the active components, ensuring prolonged therapeutic effects. PEGs improve the processability of the soft chew dough during manufacturing.

They help in achieving the right consistency for extrusion or molding, ensuring uniformity and quality in the final product. Preferably, the polyethylene glycol is PEG 3350.

Advantageously, the soft chew according to the invention comprises 1-10 wt. % of PEG having a molecular weight of 3000 to 8000 g/mol relative to the total weight of the soft chew, more preferably 3-8 wt. % of PEG having a molecular weight of 3000 to 8000 g/mol relative to the total weight of the soft chew.

In the context of the present invention, the term ‘a non-animal based palatant’ refers to any additive used to enhance the taste, flavor, aroma and palatability of the product that is free from animal-derived ingredients. This includes plant-based or synthetic flavor enhancers that are specifically designed to mimic the appealing flavors traditionally derived from animal sources. Examples of such palatants include yeast extracts, vegetable broths: hydrolyzed vegetable Proteins, plant-based flavor enhancers, synthetic flavors and dairy products. Clearly, a non-animal based palatant contains no components of animal origin.

In one embodiment, the non-animal based palatant is a vegetal-based palatant.

Such palatants are known in the art and commercially available from, for example, AFB International, Leiber, Diana Pet Foods, Lallemand and Kemin Industries.

In an embodiment, the non-animal based palatant is therefore a dry palatability enhancer containing vegetable derivatives, yeasts, sugars, nutritional additives, mineral substances, sensory additives, preservatives (citric acid, ascorbic acid), antioxidants (tocopherols).

In another embodiment, the non-animal based palatant is a dry palatability enhancer containing 9-15% protein, 0-1.5% fat, 5-11% ash, 3-7>% of water, about 0.06% calcium, about 0.20% phosphorus, about 0.03% magnesium, about 0.3% potassium, about 2.2% sodium and about 2% chloride.

Without wishing to being bound by theory, it is believed that the low amount of protein and the nature of the protein (e.g. the presence of high levels of sulfur amino-acids) contained in the non-animal based palatant influence positively the stability of milbemycin oxime and the textural properties of the soft chew when compared to an animal based palatant.

Advantageously, the soft chew according to the present invention comprises 20-40 wt. % of non-animal based palatant relative to the total weight of the soft chew, more preferably 25-35 wt. % of non-animal based palatant relative to the total weight of the soft chew.

As demonstrated in the examples, the Applicant has discovered that a non-animal based palatant provides better stability for the APIs described in the present invention compared to an animal-based palatant.

The term “disintegrant” refers to a substance that facilitates the breakup of the chewable dosage form, ensuring it disintegrates quickly and efficiently in the animal's digestive system for optimal absorption and effectiveness of the active ingredients.

Preferably, the disintegrant is croscarmellose sodium.

Advantageously, the soft chew according to the present invention comprises 5-15 wt. % of disintegrant relative to the total weight of the soft chew, more preferably 8-12 wt. % of disintegrant relative to the total weight of the soft chew.

In the sense of the present invention, a humectant refers to a substance that is hygroscopic, e.g. has the affinity to form hydrogen bonds with molecules of water, and is capable of keeping the soft chew dough moist. Humectants are well known to those skilled in the art.

In the present invention, the humectant is preferably glycerol.

Advantageously, the soft chew according to the present invention comprises 5-15 wt. % of humectant relative to the total weight of the soft chew, more preferably 8-12 wt. % of humectant relative to the total weight of the soft chew.

Advantageously, the soft chew according to the present invention comprises 1-8 wt. % of oil relative to the total weight of the soft chew, more preferably 3-5 wt. % of oil relative to the total weight of the soft chew.

Preferably, the soft chew comprises an oil that is soybean oil.

Advantageously, the soft chew according to the present invention comprises 1-8 wt. % of water relative to the total weight of the soft chew, more preferably 3-5 wt. % of water relative to the total weight of the soft chew.

It has been found that in the absence of water, the soft chew has an inadequate texture. In particular, it has an oily feel and has a tendency to crumble when held in the hand.

In a preferred embodiment, the soft chew according to the invention comprises water and oil in the same order of magnitude.

In one preferred embodiment, the ratio of water to oil is between 1:1.5 and 1.5:1, preferably the ratio of water to oil is 1:1.

In the present invention, preservatives are substances that prevent microbiological contamination and improve the stability of the formulation, including the nutraceutical and/or pharmaceutical ingredient.

Preservatives are well known in the art and can be selected depending on the active ingredient and/or formulation.

The preservative is preferably selected from acids, alcohols, phenols, parabens, sorbates, thiols, phenyl mercury salts, or any combination of any two or more thereof.

In the present invention, antioxidants are substances used to protect the active ingredient from oxidation. Some antioxidant may also act as preservatives.

Any antioxidant known in the art may be used. Preferably, the antioxidant is selected from propyl gallate, ascorbic acid and its derivatives, sodium formaldehyde sulfoxylate, malic acid, fumaric acid, editic acid, citric acid, thiols, polyphenols, sodium EDTA, sodium ascorbate, sodium metabisulfite, butylated hydroxytoluene (BHT), butylated hydroxyanisole (BHA), or natural substances such as flavanoids, tocopherols such as ascorbic acid tocopherols, carotenes, cysteine, or any combination of any two or more thereof.

In a preferred embodiment, the preservative is an antioxidant selected from beta hydroxy carboxylic acid (BHA) or butylated hydroxytoluene (BHT).

In another preferred embodiment, the soft chew comprises a preservative that is BHA, BHT or mixtures thereof.

Advantageously, the soft chew according to the invention comprises 0.01-0.2 wt. % of preservative relative to the total weight of the soft chew, more preferably 0.01-0.05 wt. % of preservative relative to the total weight of the soft chew.

In one embodiment, the soft chew according to the invention comprises:
1.1-11 wt. % of APIs relative to the total weight of the soft chew,
23-60 wt. % of fillers relative to the total weight of the soft chew,
1-10 wt. % of PEG having a molecular weight of 3000 to 8000 g/mol relative to the total weight of the soft chew,
20-40 wt. % of non-animal based palatant relative to the total weight of the soft chew,
5-15 wt. % of disintegrant relative to the total weight of the soft chew,
5-15 wt. % of humectant relative to the total weight of the soft chew,
1-8 wt. % of oil relative to the total weight of the soft chew,
1-8 wt. % of water relative to the total weight of the soft chew, and
0.01-0.2 wt. % of preservative relative to the total weight of the soft chew.

In another embodiment, the soft chew according to the invention comprises:
3-5 wt. % of APIs relative to the total weight of the soft chew,
25-35 wt. % of fillers relative to the total weight of the soft chew
3-8 wt. % of PEG having a molecular weight of 3000 to 8000 g/mol relative to the total weight of the soft chew,
25-35 wt. % of non-animal based palatant relative to the total weight of the soft chew,
8-12 wt. % of disintegrant relative to the total weight of the soft chew,
8-12 wt. % of humectant relative to the total weight of the soft chew,
3-5 wt. % of oil relative to the total weight of the soft chew,
3-5 wt. % of water relative to the total weight of the soft chew, and
0.01-0.05 wt. % of preservative relative to the total weight of the soft chew.

In another aspect, the invention relates to a soft chew, directly obtained by a cold extrusion process, said soft chew comprising as active pharmaceutical ingredients (APIs):
a) milbemycin oxime or one of its salts, and
b) praziquantel or one of its salts, said APIs being uniformly dispersed in a matrix comprising:
one or more fillers, selected from starch, sugar, microcrystalline cellulose or mixtures thereof,
a polyethylene glycol (PEG) having a molecular weight of 3000 to 8000 g/mol,
a non-animal based palatant,
a disintegrant selected from croscarmellose sodium, sodium carboxymethyl cellulose, sodium starch glycolate or mixtures thereof,
a humectant selected from glycerol, sorbitol, propylene glycol, liquid polyethylene glycol, liquid paraffin or mixtures thereof,
an oil selected from canola oil, corn oil, soybean oil, safflower oil, cottonseed oil, soybean oil, olive oil or mixtures thereof,
water, and
preservatives, said cold extrusion process comprising the steps of
i. : mixing directly and intimately the ingredients, in a blinder and/or an extruder wherein neither the milbemycin oxime or its salts, nor the praziquantel or its salts is coated, granulated or physically separated from the other APIs within the blend, at a temperature not exceeding 60° C.,
ii. : extruding the final blend by subjecting the mixture to high mechanical pressure to form a cohesive matrix without thermal cooking;
iii. extruding and forming the mixture into a soft chew form, said soft chew exhibiting a tender texture and retaining the active properties of the APIs, characterized by a water content of up to 10% and an active ingredient activity of at least 95% relative to initial values prior to extrusion.

In a preferred embodiment, the water content of the soft chew according to the invention is up to 10%, preferably up to 9%, preferably up to 8%, preferably up to 7%, preferably up to 6% and more preferably of about 7 to 8% relative to initial values prior to extrusion.

In another aspects, the invention relates to a cold extrusion process for the manufacture of said extruded soft chew, comprising the following steps:
i. mixing, in a blinder and/or an extruder directly and intimately at least the following ingredients:
a) milbemycin oxime or one of its salts, and b) praziquantel or one of its salts,
one or more fillers, selected from starch, sugar, microcrystalline cellulose or mixtures thereof,
a polyethylene glycol having a molecular weight of 3000 to 8000 g/mol,
a non-animal based palatant,
a disintegrant selected from croscarmellose sodium, sodium carboxymethyl cellulose, sodium starch glycolate or mixtures thereof,
a humectant selected from glycerol, sorbitol, propylene glycol, liquid polyethylene glycol, liquid paraffin or mixtures thereof,
an oil selected from canola oil, corn oil, soybean oil, safflower oil, cottonseed oil, soybean oil, olive oil or mixtures thereof,
water, and
preservatives,
   wherein neither the milbemycin oxime or its salts, nor the praziquantel or its salts is coated, granulated or physically separated from the other APIs within the blend
ii. extruding the final blend, and
iii. forming a soft chew.

Without wishing to be bound by theory, it is believed that the fact that the manufacturing of the soft chews is carried out by a particular process, namely extrusion, and specifically cold extrusion (which involves processing the ingredients at low temperatures to avoid or limit heat-induced degradation and preserve the active ingredients), is an important element in the invention.

Other manufacturing processes for soft chews, such as molding or compression, are considered to be less suitable because of process feasibility and results in terms of the textural properties of the soft chew.

In an advantageous embodiment, in step i. of mixing of the process according to the invention, the solids are first mixed together, then the liquids are added.

In a more advantageous embodiment, the process according to the invention comprises the following steps:
pre-blend of the APIs with selected excipients, preferably with starch and iron oxide,
pre-blend of the other dry excipients,
preparation of the liquid phase mixture,
add pre-blend of the APIs in Pre-blend of the other dry excipients,
add liquid phase of excipients on dry blend during mixing,
final blend holding storage, and
extrusion of the final blend and chew forming.

Preferably, step i. of mixing is performed at room temperature, i.e. without using an external source of heat. Carrying out the process at room temperature makes it possible to use heat sensitive nutraceutical ingredients and/or pharmaceutical active substances without degradation.

Preferably, step i. of mixing is performed at a temperature preferably not exceeding 60° C., preferably not exceeding 50° C., preferably not exceeding 45° C.; more preferably not exceeding 40° C.; and more preferably not exceeding 35° C., and more preferably not exceeding 30° C.; and more preferably not exceeding 27° C.; and more preferably not exceeding 25° C.

Step i. and step ii. may occur simultaneously or separately, depending on the machines used. In one embodiment, mixing is performed prior to forming the soft chews.

In a first alternative, mixing is performed in a mixer until a homogeneous dough is obtained. It has been found that mixers conventionally employed to manufacture soft chews (e.g. the ones conventionally used in the pharmaceutical and food industries) did not make it possible to obtain a homogeneous dough because the mixture was too viscous if not heated during the mixing (to prevent a possible degradation of the actives). Therefore, mixers equipped with a Z-arm, a T-arm, or a S-arm, preferably a Z-arm, more conventionally used in the plastics industry, were preferred.

In a second alternative, mixing is performed in an extruder. All the ingredients of the soft chew may be charged in an inlet port, mixed in the extruder during conveyance to the discharge port or the solid ingredients charged in the inlet port and the liquids added through additional inlet ports located on the barrel of the extruder to provide a dough.

The dough obtained in the mixer, or the extruder is then formed into a soft chew of appropriate size and weight.

In one embodiment, a die-cutting process can be placed directly at the end of the extrusion line. This method eliminates the need for post-extrusion cutting and shaping operations, streamlining the overall manufacturing process. By strategically placing a die at the exit of the extruder, the extruded material can be immediately cut into desired shapes and sizes, such as cubes, sticks, or irregular shapes.

The soft chew may be formed by any method known in the art. A variety of forming equipment may be utilized in the invention, for example, the molding machines disclosed in U.S. Pat. Nos. 3,486,186; 3,887,964; 3,952,478; 4,054,967; 4,097,961; 4,182,003; 4,334,339; 4,338,702; 4,343,068; 4,356,595; 4,372,008; 4,535,505; 4,597,135; 4,608,731; 4,622,717; 4,697,308; 4,768,941; 4,780,931; 4,818,446; 4,821,376; 4,872,241; 4,975,039; 4,996,743; 5,021,025; 5,022,888; 5,655,436; and 5,980,228, and in US patent application US 2014/0141055 or tablet presses. Particularly contemplated are the Formax F6 molding machine and the rotary molding machines, including described in US 2014/0141055 (MFT-200 of Kriger & Salecker, Bad Schwartau).

Preferably, in the processes for manufacturing the soft chews described above, in step i. of mixing, the solids are first mixed together then the liquids are added. More preferably, the at least one oil and water are added sequentially to the solids. In one particular embodiment, the at least one oil is added first to the solids then water is added. It has been observed that addition of the at least one oil before the addition of water to the solid ingredients results in a lesser exudation of oil from the soft chew.

The process may additionally comprise an additional curing step after step iii, which is a fourth step iv. of cooling and maturation, said step being preferably performed at room temperature for at least 15 minutes.

In the sense of the present invention, a curing step refers to a step of cooling and maturation aiming at favoring the phenomena of starch cross-linking and network reorganization to get into a more stable state and to avoid the chews to stick with each other.

The curing step is preferably performed by leaving the soft chew on a planar surface for a sufficient time after formation of the soft chews, to let it cool and mature. It has been found that neither heating nor cooling accelerates the maturation of the soft chews. The curing step may therefore be performed at room temperature (e.g. between 15 and 30° C., in the cold (e.g. between −15 and 15° C.) or at hot temperature (e.g. between 30 and 60° C.). The curing time may be readily determined by the person skilled in the art. Preferably, the soft chew is cooled and matured for a period between 15 minutes and 24 hours, more preferably between 1 hour and 12 hours, in particular about 1 hour to 3 hours.

After curing, the soft chews can be packed into bottles or containers. Said containers can be glass containers, glass bottles, plastic (for example HDPE) bottles or plastic (for example HDPE) containers.

The present invention therefore also concerns a process as described above, comprising a step v. of packaging the soft chews.

In another aspect, the soft chew according to the present invention is used as a medicament for non-human animals, preferably dogs and/or cats.

More particularly, the soft chew according to the present invention is used in the treatment and/or prevention of parasitic infestations in pets.

By “treating” or “treat” or “treatment” is intended the administration of a composition of the invention to an animal that has a parasitic infestation for the eradication of the parasite or the reduction of the number of the parasites infesting the animal undergoing treatment. It is noted that the compositions of the invention may be used to prevent such a parasitic infestations.

More particularly, the soft chew according to the present invention is used for the treatment and/or prevention of mixed infestations by nematodes and adult cestodes.

More particularly, the soft chew according to the present invention is used for the treatment and/or prevention of infections caused by mixed infestations by nematodes and adult cestodes.

More particularly, the soft chew according to the present invention is used for the treatment and/or prevention of mixed infections caused by nematodes Ancylostoma caninum, Ancylostoma tubaeforme toxocara canis, toxocara cati, toxascaris leonine, trichuris vulpis, crenosoma vulpis, angiostrongylus vasorum, thelazia callipaeda, Dirofilaria immitis and adult cestodes: Dipylidium caninum, Taenia spp., Echinococcus spp., Echinococcus multilocularis, Mesocestoides spp.

In another aspect, the soft chew of the present invention comprises an API that may be included in the composition to deliver a dose of at least 0.05 mg/kg to 5 mg/kg, 0.5 mg/kg to 5 mg/kg, 2 mg/kg to 5 mg/kg of body weight (BW) of the animal.

For example, the soft chew of the present invention comprises milbemycin oxime or one of its salts at a dose of at least 0.5 mg/kg of BW and praziquantel or one of its salts at a dose of at least 5 mg/kg of BW, milbemycin oxime or one of its salts at a dose of at least 2 mg/kg of BW and praziquantel or one of its salts at a dose of at least 5 mg/kg of BW.

In another aspect, the soft chew of the present invention comprises an API that may be included in the composition at a dose of at least 1 mg to 125 mg.

In another aspect, the soft chew of the present invention comprises an API that may be included in the composition at a dose of at least 1 mg, 1.5 mg, 2 mg, 2.5 mg, 3 mg, 4 mg, 5 mg, 10 mg, 12.5 mg, 16 mg, 25 mg, 40 mg, 125 mg.

For example, the soft chew of the present invention comprises milbemycin oxime or one of its salts at a dose of at least 2.5 mg and praziquantel or one of its salts at a dose of at least 25 mg, milbemycin oxime or one of its salts at a dose of at least 4 mg and praziquantel or one of its salts at a dose of at least 10 mg, milbemycin oxime or one of its salts at a dose of at least 16 mg and praziquantel or one of its salts at a dose of at least 40 mg, milbemycin oxime or one of its salts at a dose of at least 12.5 mg and praziquantel or one of its salts at a dose of at least 125 mg.

The compositions of the invention can be in a variety of forms suitable for different forms of administration including, but are not limited to oral formulations.

In another aspect, the soft chew of the present invention is administered once per day, once per week, once per month, once per year.

### EXAMPLES

### Example 1: Binary Mixture Preliminary Stability Study

The objective of this study was to determine the most stable palatants with respect to Milbemycin oxime. The main objective being a gain of information on subsequent prototype tests, crash tests and analyzes.

The study was performed as follows:
the content of milbemycin oxime was measured at TO by UHPLC;
milbemycin oxime and a palatant in a 1:1 weight to weight ratio (w/w) were thoroughly mixed to give a powder mixture;
said mixture was placed in a dry oven at 50 days for 60 days (in brown glass bottles); and
the content of milbemycin oxime was measured at T0+60 days (T60) by UHPLC.

Information available on the different palatants are given in table 1.

TABLE 1
Content of the palatants

| | | | | Water | | |
|---|---|---|---|---|---|---|
| | | Fat | Protein | content | Ash | Other |
| Type | Description | (%) | (%) | (%) | (%) | (%) |
| Animal | Poultry Liver | 10-14 | 52-58 | 3-7 | 5.5-9.5 | |
| based | Powder | | | | | |
| palatant | | | | | | |
| Non-animal | Spray dried | 0.50 | 70% | <15 | 0 | |
| based | yeast extract | | | | | |
| palatant | | | | | | |
| Non-animal | Yeast, sugars, | 1-3 | 32-38 | 3-7 | 17-23 | 0.05 Ca; |
| based | minerals, | | | | | 3 P; |
| palatant | vegetable | | | | | 2 Cl; |
| | derivatives. | | | | | 1 K |
| | Preservatives, | | | | | 0.2 Mg; |
| | antioxidants. | | | | | 6.5 Na |
| Non-animal | Brewers Yeast | 0 | 50 | 5 | 6-8 | |
| based | Autolysate | | | | | |
| palatant | | | | | | |
| Non-animal | Dried yeast | NA | 63-75 | <6 | | <1 NaCl |
| based | extract powder | | | | | |
| palatant | | | | | | |
| Non-animal | Brewers' Yeast | NA | NA | NA | NA | NA |
| based | dried | | | | | |
| palatant | | | | | | |
| Non-animal | Meat Flavor | NA | NA | NA | NA | NA |
| based | | | | | | |
| palatant | | | | | | |
| Non-animal | Cookies Flavor | NA | NA | NA | NA | NA |
| based | | | | | | |
| palatant | | | | | | |

The stability of milbemycin oxime in table 2 is expressed as a percentage, corresponding to the content of milbemycin oxime at T60/the content of milbemycin oxime at T0.

TABLE 2
Content of milbemycin oxime

| | | % of milbemycin |
|---|---|---|
| Product | Formula | oxime (T60/T0) |
| Milbemycin oxime/ | 1:1 w/w mixture | 99.4 |
| Spray dried yeast | | |
| extract | | |
| Milbemycin oxime/ | 1:1 w/w mixture | 98.2 |
| Poultry liver | | |
| powder | | |
| Milbemycin oxime/ | 1:1 w/w mixture | 100 |
| Yeast, sugars, | | |
| minerals, vegetable | | |
| derivatives. | | |
| Preservatives, | | |
| antioxidants. | | |
| Milbemycin | 1:1 w/w mixture | 98.7 |
| oxime/Cookies | | |
| flavor | | |
| Milbemycin oxime/ | 1:1 w/w mixture | 100 |
| Meat flavor | | |
| Milbemycin oxime/ | 1:1 w/w mixture | 100 |
| Dried yeast extract | | |
| powder | | |
| Milbemycin oxime/ | 1:1 w/w mixture | 100 |
| Brewers Yeast | | |
| Autolysate | | |

### Example 2: Formulation Assessment

The objective of this study was to determine physico-chemical properties of different soft chews. The main objective is to determine the best palatant between an animal based palatant (formulation B: poultry liver powder) and a non-animal based palatant (formulation A: yeast, sugars, minerals, vegetable derivatives, preservatives, antioxidants). Information about the composition of both tested soft chews is presented in table 3.

TABLE 3
Formulations of the soft chews

| | Formula A (non- | |
|---|---|---|
| | animal based | Formula B (animal |
| Ingredient | palatant) | based palatant) |
| Milbemycin | 0.38 | 0.38 |
| Praziquantel | 3.8 | 3.8 |
| Total APIs | 4.18 | 4.18 |
| Corn Starch | 20 | 20 |
| Confectioners Sugar | 5 | 5 |
| PEG 3350 NF Powder | 5 | 5 |
| Microcristalline | 5.83 | 5.83 |
| Cellulose PH102 | | |
| Yeast, sugars, | 30 | |
| minerals, vegetable | | |
| derivatives. | | |
| preservatives, | | |
| antioxidants | | |
| Poultry liver | | 30 |
| powder | | |
| Croscarmellose | 10 | 10 |
| Sodium | | |
| Glycerin | 10.35 | 10.35 |
| Soybean Oil | 4 | 4 |
| Purified water | 4 | 4 |
| BHA | 0.04 | 0.04 |
| Sodium Chloride | 1.5 | 1.5 |
| Iron Oxide Blend | 0.1 | 0.1 |
| total | 100 | 100 |

The soft chews were manufactured by extrusion (Bonnot FTP 1 Lab Extruder) using the following process:
pre-blend of the APIs with selected excipients, preferably with starch and iron oxide;
pre-blend of the other dry excipients;
preparation of the liquid phase mixture;
add pre-blend of the APIs in Pre-blend of the other dry excipients;
add liquid phase of excipients on dry blend during mixing;
final blend holding storage; and
extrusion of the final blend and chew forming.

The soft chews were then subjected to different analyses to evaluate the stability of the actives and their textural properties at different time points. The following properties were analysed:
milbemycin oxime content;
praziquantel content;
hardness; and
disintegration time.

The results are presented in table 4.

TABLE 4
Physico-chemical results

| | | Formula A | Formula B |
|---|---|---|---|
| Milbemycin oxime | T0 | 100 | 100 |
| content | 3 months at | 95.8 | 88.6 |
| (%/theory) | 40° C., 75% | | |
| | RH | | |
| | 6 months at | 94.6 | 78.4 |
| | 40° C., 75% | | |
| | RH | | |
| Praziquantel | T0 | 100 | 100 |
| content | 3 months at | 100 | 99.2 |
| (%/theory) | 40° C., 75% | | |
| | RH | | |
| | 6 months at | 100 | 95.5 |
| | 40° C., 75% | | |
| | RH | | |
| Hardness | T0 | 34.5 | 46.0 |
| (Newton) | 3 months at | 61.2 | 82.0 |
| | 25° C., 60% | | |
| | RH | | |
| | 3 months at | 62.7 | 83.2 |
| | 40° C., 75% | | |
| | RH | | |
| Disintegration | T0 | 1800 | 1500 |
| time (Second) | 3 months at | 1500 | 2100 |
| | 25° C., 60% | | |
| | RH | | |
| | 3 months at | 1800 | 2700 |
| | 40° C., 75% | | |
| | RH | | |
| | 6 months at | 1455 | — |
| | 25° C., 60% | | |
| | RH | | |
| Total visible | T0 | — | — |
| impurities at | 3 months at | 0.8 | 1.7 |
| 240 nm (%/MBO) | 40° C., 75% | | |
| | RH | | |
| | 6 months at | 1.5 | 4.3 |
| | 40° C., 75% | | |
| | RH | | |

One notable impact of the animal based palatant is on the stability of milbemycin oxime: at 40° C./75% RH (Relative Humidity), the content in milbemycin oxime drops significantly and more importantly below acceptable levels. In contrast, only a slight and acceptable degradation of milbemycin oxime is observed with the non-animal based palatant. The content in milbemycin decreases by 5,4% in 6 months (at 40° C., 75 RH) in the non-animal based palatant, in other words 4 times less than in the animal based one.

The content in praziquantel remains constant for 6 months with the non-animal based palatant. We observe a slight decrease in the praziquantel content with the animal-based palatant, especially after 6 months at 40° C./75% RH. Praziquantel appears much more stable than milbemycin oxime both with animal based and non-animal based palatants.

The type of palatant also has a strong influence on the textural properties of the soft chew: while both palatants provide acceptable texture and disintegration times, the animal based palatant induces a significant hardening after three months. It also induces a significant modification of the disintegration time. Both characteristics are unacceptable for a soft chew.

Finally, the animal based palatant induces an increase in the total visible impurities. After 6 months at 40° C./75% RH, it is three times higher with the animal based palatant than with the non-animal based palatant.

The non-animal based palatant increases the quality of the soft chew, especially compared with the animal based one.

### Example 3: Appetence Test

The purpose of this study was to determine and to compare the appetences of the soft chews with the non-animal based palatant and with the animal based palatant presented in table 3.

Both palatants were prepared according to the process presented in example 2. Soft chews were presented to 30 dogs at T0, and the capture were studied, in other words the number of dogs having gripped the soft chew in their mouths (directly from bowl or only if the soft chew was given by a human hand).

The results are presented in the FIGURE.

The results are equivalent: 25 out of 30 dogs took the soft chew with the non-animal based palatant and 26 out of 30 dogs took the soft chew with the animal based palatant. The results are not significantly different.

It seems there is no appetence difference between the animal based and the non-animal based palatants.

However, this study was carried out at T0. Example 2 shown that hardness and disintegration time were decreasing faster in the soft chew with the animal based palatant than in the soft chew with the non-animal based palatant. It appears that the drop of the textural properties induced by the animal based palatant could have an influence on the appetence of the soft chew over time. As shown in example 2, the textural properties of the soft chew with the non-animal based palatant are better.

In conclusion, the Applicant has been able to demonstrate that the soft chew with a non- animal based palatant has the same appetence compared to the animal based palatant soft chew; and at the same time, in the non-animal based palatant soft chew, the APIs are more stable than when an animal based palatant is added.

1. A veterinary extruded soft chew comprising:
as active pharmaceutical ingredients (APIs):
a) milbemycin oxime or one of its salts, and
b) praziquantel or one of its salts, said APIs being uniformly dispersed in a matrix comprising:
one or more fillers, selected from the group consisting of starch, sugar, microcrystalline cellulose, and mixtures thereof,
a polyethylene glycol (PEG) having a molecular weight of 3000 to 8000 g/mol,
a non-animal based palatant,
a disintegrant selected from the group consisting of croscarmellose sodium, sodium carboxymethyl cellulose, sodium starch glycolate, and mixtures thereof,
a humectant selected from the group consisting of glycerol, sorbitol, propylene glycol, liquid polyethylene glycol, liquid paraffin, and mixtures thereof,
an oil selected from the group consisting of canola oil, corn oil, soybean oil, safflower oil, cottonseed oil, soybean oil, olive oil, and mixtures thereof,
water, and
a preservative,
wherein neither the milbemycin oxime or its salts, nor the praziquantel or its salts is coated, granulated or physically separated from the other APIs within the matrix and, wherein the soft chew does not comprise any palatant of animal origin.

2. The soft chew according to claim 1, comprising:
1.1-11 wt. % of APIs relative to the total weight of the soft chew,
23-60 wt. % of fillers relative to the total weight of the soft chew,
1-10 wt. % of PEG having a molecular weight of 3000 to 8000 g/mol relative to the total weight of the soft chew,
20-40 wt. % of non-animal based palatant relative to the total weight of the soft chew,
5-15 wt. % of disintegrant relative to the total weight of the soft chew,
5-15 wt. % of humectant relative to the total weight of the soft chew,
1-8 wt. % of oil relative to the total weight of the soft chew,
1-8 wt. % of water relative to the total weight of the soft chew, and
0.01-0.2 wt. % of preservative relative to the total weight of the soft chew.

3. The soft chew according to claim 2, comprising:
3-5 wt. % of APIs relative to the total weight of the soft chew,
25-35 wt. % of fillers relative to the total weight of the soft chew
3-8 wt. % of PEG having a molecular weight of 3000 to 8000 g/mol relative to the total weight of the soft chew,
25-35 wt. % of non-animal based palatant relative to the total weight of the soft chew,
8-12 wt. % of disintegrant relative to the total weight of the soft chew,
8-12 wt. % of humectant relative to the total weight of the soft chew,
3-5 wt. % of oil relative to the total weight of the soft chew,
3-5 wt. % of water relative to the total weight of the soft chew, and
0.01-0.05 wt. % of preservative relative to the total weight of the soft chew.

4. The soft chew according to claim 1, wherein the one or more fillers is a mixture of starch, sugar and microcrystalline cellulose.

5. The soft chew according to claim 4, wherein the fillers comprise 19-23 wt. % of starch of total weight of the soft chew, 3-6 wt. % of sugar of total weight of the soft chew and 3-6 wt. % microcrystalline cellulose of total weight of the soft chew.

6. The soft chew according to claim 1, wherein the polyethylene glycol is PEG 3350.

7. The soft chew according to claim 1, wherein the disintegrant is croscarmellose sodium.

8. The soft chew according to claim 1, wherein the humectant is glycerol.

9. The soft chew according to claim 1, wherein the preservative is selected from BHA or BHT.

10. The soft chew according to claim 1, wherein the soft chew is a non-human animal soft chew.

11. The soft chew according to claim 10, wherein the soft chew is configured for a dog.

12. The soft chew according to claim 10, wherein the soft chew is configured for a cat.

13. A soft chew, directly obtained by a cold extrusion process, said soft chew comprising as active pharmaceutical ingredients (APIs):
a) milbemycin oxime or one of its salts, and
b) praziquantel or one of its salts,
said APIs being uniformly dispersed in a matrix comprising:
one or more fillers, selected from the group consisting of starch, sugar, microcrystalline cellulose, and mixtures thereof,
a polyethylene glycol (PEG) having a molecular weight of 3000 to 8000 g/mol,
a non-animal based palatant,
a disintegrant selected from the group consisting of croscarmellose sodium, sodium carboxymethyl cellulose, sodium starch glycolate, and mixtures thereof,
a humectant selected from the group consisting of glycerol, sorbitol, propylene glycol, liquid polyethylene glycol, liquid paraffin, and mixtures thereof,
an oil selected from the group consisting of canola oil, corn oil, soybean oil, safflower oil, cottonseed oil, soybean oil, olive oil, and mixtures thereof,
water, and
preservatives,
said cold extrusion process comprising the steps of:
i. mixing directly and intimately the ingredients, in a blender and/or an extruder wherein neither the milbemycin oxime or its salts, nor the praziquantel or its salts is coated, granulated or physically separated from the other APIs within the blend, at a temperature not exceeding 60° C.,
ii. extruding the final blend by subjecting the mixture to high mechanical pressure to form a cohesive matrix without thermal cooking;
iii. extruding and forming the mixture into a soft chew form, said soft chew exhibiting a tender texture and retaining the active properties of the APIs,
characterized by a water content up to 10% and an active ingredient activity of at least 95% relative to initial values prior to extrusion.

14. A soft chew, directly obtained by a cold extrusion process, said soft chew comprising as active pharmaceutical ingredients (APIs):
a) milbemycin oxime or one of its salts, and
b) praziquantel or one of its salts,
said APIs being uniformly dispersed in a matrix comprising:
one or more fillers, selected from the group consisting of starch, sugar, microcrystalline cellulose, and mixtures thereof,
a polyethylene glycol (PEG) having a molecular weight of 3000 to 8000 g/mol,
a non-animal based palatant,
a disintegrant selected from the group consisting of croscarmellose sodium, sodium carboxymethyl cellulose, sodium starch glycolate, and mixtures thereof,
a humectant selected from the group consisting of glycerol, sorbitol, propylene glycol, liquid polyethylene glycol, liquid paraffin, and mixtures thereof,
an oil selected from the group consisting of canola oil, corn oil, soybean oil, safflower oil, cottonseed oil, soybean oil, olive oil, and mixtures thereof,
water, and
preservatives,
said cold extrusion process comprising the steps of:
i. mixing directly and intimately the ingredients, in a blender and/or an extruder wherein neither the milbemycin oxime or its salts, nor the praziquantel or its salts is coated, granulated or physically separated from the other APIs within the blend, at a temperature not exceeding 60° C.,
ii. extruding the final blend by subjecting the mixture to high mechanical pressure to form a cohesive matrix without thermal cooking;
iii. extruding and forming the mixture into a soft chew form, said soft chew exhibiting a tender texture and retaining the active properties of the APIs,
characterized by a water content of about 7 to 8% and an active ingredient activity of at least 95% relative to initial values prior to extrusion.

15. A cold extrusion process for the manufacture of soft chew according to claim 1, comprising the following steps:
i. mixing, in a blender and/or an extruder, directly and intimately at least the following ingredients:
milbemycin oxime or one of its salts,
praziquantel or one of its salts,
one or more fillers, selected from the group consisting of starch, sugar, microcrystalline cellulose, and mixtures thereof,
a polyethylene glycol having a molecular weight of 3000 to 8000 g/mol,
a non-animal based palatant,
a disintegrant selected from the group consisting of croscarmellose sodium, sodium carboxymethyl cellulose, sodium starch glycolate, and mixtures thereof,
a humectant selected from the group consisting of glycerol, sorbitol, propylene glycol, liquid polyethylene glycol, liquid paraffin, and mixtures thereof,
an oil selected from the group consisting of canola oil, corn oil, soybean oil, safflower oil, cottonseed oil, soybean oil, olive oil, and mixtures thereof,
water, and
preservatives,
wherein neither the milbemycin oxime or its salts, nor the praziquantel or its salts is coated, granulated or physically separated from the other APIs within the blend
ii. extruding the final blend, and
iii. forming a soft chew.

16. A method of treatment, the method comprising:
providing a soft chew according to claim 1 to a non-human animal in need thereof, and
orally administering the soft chew.

17. The method according to claim 16, wherein the non-human animal is a dog.

18. The method according to claim 16, wherein the non-human animal is a cat.
